(19) United States
(12) Patent Application Publication
(10) Pub. No.: US 2024/0148923 A1
(43) Pub. Date: May 9, 2024

(54) STERILIZATION DEVICE

(71) Applicant: SL Corporation, Daegu (KR)

(72) Inventors: Hyun Woo Choi, Gyeongsan-si (KR); Sang Hyoung Lee, Gyeongsan-si (KR); Woo Yeong Son, Gyeongsan-si (KR); Jong Woon Kim, Gyeongsan-si (KR); Hae Ryun Lee, Gyeongsan-si (KR); Man Young Chun, Gyeongsan-si (KR); Dong Hwan Sin, Gyeongsan-si (KR)

(21) Appl. No.: 18/410,976

(22) Filed: Jan. 11, 2024

Related U.S. Application Data
(63) Continuation of application No. PCT/KR2022/005028, filed on Apr. 7, 2022.

(30) Foreign Application Priority Data
Jul. 22, 2021 (KR) 10-2021-0096425

(51) Int. Cl. A61L 2/10

(52) U.S. Cl. CPC A61L 2/10; A61L 2202/16

(57) ABSTRACT

A sterilization device includes a housing; a sterilization unit which emits germicidal light for sterilization of at least one target area to be sterilized; and a drive unit which moves at least a part of the sterilization unit between the outside and inside of the housing through an opening provided in the housing.

### CROSS-REFERENCE TO RELATED APPLICATIONS

This application is a Continuation of International Application No. PCT/KR2022/005028 filed Apr. 7, 2022, which claims priority from Korean Application No. KR 10-2021-0096425 filed Jul. 22, 2021. The aforementioned applications are incorporated herein by reference in their entireties.

### TECHNICAL FIELD

The present disclosure relates to a sterilization device, and more particularly, to a sterilization device capable of facilitating sterilization for sterilization target areas.

### RELATED ART

Typically, public transportation, such as buses and taxis, or multi-purpose facilities, such as department stores and discount stores, involve frequent hand contact by numerous individuals. In cases where various germs and foreign substances adhere to such contact surfaces, there is a significant risk of rapid transmission thereof to many people. Thus, meticulous management of hygiene is essential.

To address this issue, efforts have been made to disinfect areas frequently touched by people using chemicals. However, chemical sterilization results in continuous consumption of chemicals, leading to increased costs. Accordingly, sterilization using light, such as ultraviolet (UV) light, is recently being used instead of chemicals to disinfect contact surfaces.

When using light for sterilization, it is necessary to ensure that the sterilization light is applied to the entire sterilization target area. In this case, multiple light sources need to be provided.

Furthermore, when there are multiple sterilization target areas, it is required to install separate light sources for the application of the sterilization light in each area. This imposes limitations on achieving sterilization for various target areas. Additionally, reducing the number of light sources can lead to an increase in the required sterilization time to satisfy the sterilization efficiency.

Therefore, there is a demand for a solution that allows easy sterilization of various target areas with a simple setup while reducing the required sterilization time to meet sterilization efficiency.

### SUMMARY

Aspects of the present disclosure provide a sterilization device capable of enabling sterilization for a variety of targeted regions.

However, aspects of the present disclosure are not restricted to those set forth herein. The above and other aspects of the present disclosure will become more apparent to one of ordinary skill in the art to which the present disclosure pertains by referencing the detailed description of the present disclosure given below.

A sterilization device according to an embodiment of the present disclosure may include a housing; a sterilization unit that irradiates sterilization light to at least one sterilization target area; and a driving unit that moves the sterilization unit between exterior and interior of the housing via an opening provided in the housing.

The sterilization unit may be moved in a vertical direction through the opening, which is formed on a top surface of the housing. The sterilization unit may include at least one light source to generate the sterilization light and a mounting part in which the at least one light source is mounted. The at least one light source may include a plurality of light sources mounted in the mounting part to be arranged in a circumferential direction centered on an axis that is parallel with a direction in which the sterilization unit is moved. Some of the plurality of light sources may have a different spacing therebetween from other light sources.

The sterilization unit may further include at least one reflector to reflect the sterilization light generated from the at least one light source to the at least one sterilization target area. The at least one reflector may have different shapes depending on at least one of an irradiation direction or an irradiation area of the reflected sterilization light.

The driving unit may include an actuator, a support bracket coupled to the sterilization unit, and a gear part that transmits power from the actuator to the support bracket. As the support bracket moves, the sterilization unit coupled to the support bracket may move. The support bracket may include at least one guide rib movably inserted in at least one guide rail, which is formed in the housing.

The sterilization device may further include a pressurizing member elastically deformed by the at least one guide rib. A first end portion of the pressurizing member may be coupled to the housing, and a second end portion of the pressurizing member may exert pressure to the at least one guide rib to allow the at least one guide rib to contact an inner surface of the at least one guide rail. The sterilization device may further include an elastic part formed to extend from the at least one guide rib, and an end portion of the elastic part may be elastically deformed by an inner surface of the at least one guide rail.

The sterilization device may further include an illumination unit installed on one side of the sterilization unit to form an illumination image having a predetermined shape. The illumination unit may include at least one light-emitting element that emits light, and a light-transmitting cover that forms the illumination image by transmitting the light emitted from the at least one light-emitting element therethrough. The at least one light-emitting element may include a light source part and a light guide part that guides light incident upon an incident portion thereof from the light source part to be emitted through an emitting portion thereof.

The light guide part may further include a transmitting portion for transmitting the incident light from the incident portion to the emitting portion. The light guide part may further include a reflecting portion that reflects the light transmitted by the transmitting portion toward the emitting portion.

The at least one light-emitting element may include a plurality of light-emitting elements, and the plurality of light-emitting elements may be turned on simultaneously or sequentially in a predetermined sequence.

The sterilization light may have a wavelength in a UV-C range. For example, the wavelength of the sterilization light may be between about 222 nm and about 280 nm.

Other specific details of the present disclosure are included in the detailed description and drawings.

The aforementioned sterilization device of the present disclosure may provide one or more effects as follows.

As multiple light sources are provided for irradiating the irradiation unit with sterilization light in different directions, various target areas may be sterilized more easily.

Furthermore, as the sterilization unit is withdrawn inside or exposed outside the housing depending on whether sterilization is to be performed or not, the space occupied by the sterilization unit when sterilization is not performed can be reduced, thereby expanding the interior space of the vehicle.

Moreover, as sterilization is performed in accordance with predefined settings when all of a plurality of sterilization conditions are met concurrently, adverse effects on humans or issues with the vehicle that may arise due to sterilization can be prevented or minimized.

It should be noted that the effects of the present disclosure are not limited to those described above, and other effects of the present disclosure will be apparent from the following description.

### BRIEF DESCRIPTION OF DRAWINGS

FIG. 1 is a perspective view illustrating the exterior of a sterilization device according to an embodiment of the present disclosure;

FIG. 2 is a side view illustrating the exterior of the sterilization device according to an embodiment of the present disclosure;

FIG. 3 is a perspective view of the sterilization device according to an embodiment of the present disclosure;

FIG. 4 is a side view of the sterilization device according to an embodiment of the present disclosure;

FIG. 5 is a perspective view of a housing according to an embodiment of the present disclosure;

FIG. 6 is a perspective view of a support bracket movably positioned within the housing according to an embodiment of the present disclosure;

FIG. 7 is a cross-sectional view of a pressurizing member according to an embodiment of the present disclosure;

FIG. 8 is a cross-sectional view of an elastic part formed in the support bracket according to an embodiment of the present disclosure;

FIG. 9 is a side view of a sterilization unit moved by a driving unit according to an embodiment of the present disclosure;

FIG. 10 is a side view of the sterilization unit positioned to be exposable on the outside of the housing according to an embodiment of the present disclosure;

FIG. 11 is a plan view of an illumination unit according to an embodiment of the present disclosure;

FIG. 12 is an exploded perspective view of the illumination unit according to an embodiment of the present disclosure;

FIG. 13 is a cross-sectional view of the illumination unit according to an embodiment of the present disclosure;

FIG. 14 is a cross-sectional view of a light-emitting element according to an embodiment of the present disclosure;

FIGS. 15 and 16 are schematic views of illumination images formed by the illumination unit according to an embodiment of the present disclosure;

FIG. 17 is a block diagram of a sterilization system according to an embodiment of the present disclosure; and

FIG. 18 is a flowchart illustrating an operating method of the sterilization system according to an embodiment of the present disclosure.

### DETAILED DESCRIPTION

The advantages and features of the present disclosure, as well as methods for achieving them, will become clear with reference to the detailed examples provided in the accompanying drawings. However, it should be noted that the present disclosure is not limited to the embodiments disclosed herein but can be implemented in various other forms. These embodiments are provided to ensure full disclosure of the present disclosure to those skilled in the art to which it pertains, and the disclosure is defined solely by the scope of the claims. Throughout the entire specification, the same reference numerals denote the same components.

Therefore, in some embodiments, well-known process steps, well-known structures, and well-known technologies are not specifically described to avoid any ambiguity in the interpretation of the present disclosure.

The terminology used in this specification is for describing the embodiments and is not intended to limit the present disclosure. In this specification, unless otherwise specified, the singular form includes the plural form. The terms “comprises” and/or “comprising” used in the specification are intended to indicate that the named components, steps, operations, and/or elements do not exclude the presence or addition of one or more other components, steps, operations, and/or elements beyond those mentioned. Moreover, “and/or” encompasses each of the mentioned items and all possible combinations thereof.

Furthermore, the embodiments described in this specification will be explained with reference to exemplary sectional and/or schematic views of the present disclosure. Therefore, the form of the embodiments of the present disclosure may be altered by manufacturing techniques and/or permissible tolerances, among other factors. Hence, the embodiments of the present disclosure include variations in shape that may be generated according to the manufacturing process, and it should be noted that the components in each drawing of the present disclosure may be slightly enlarged or reduced for the convenience of explanation. Throughout the entire specification, the same reference numerals denote the same components.

A sterilization device according to an embodiment of the present disclosure will hereinafter be described with reference to the accompanying drawings.

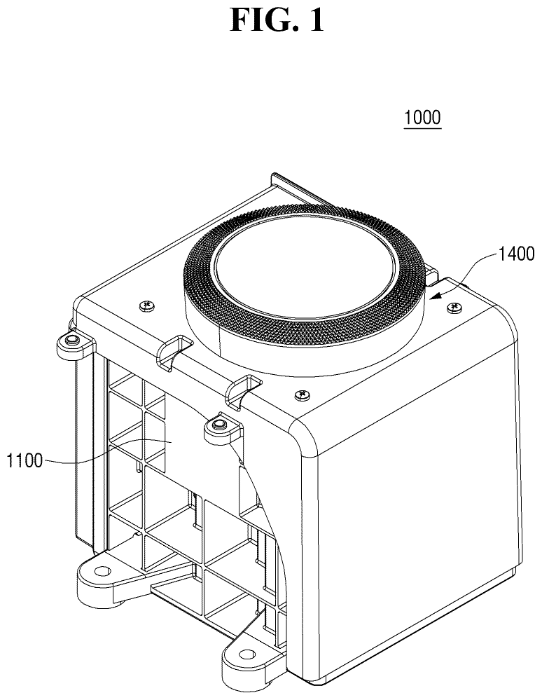

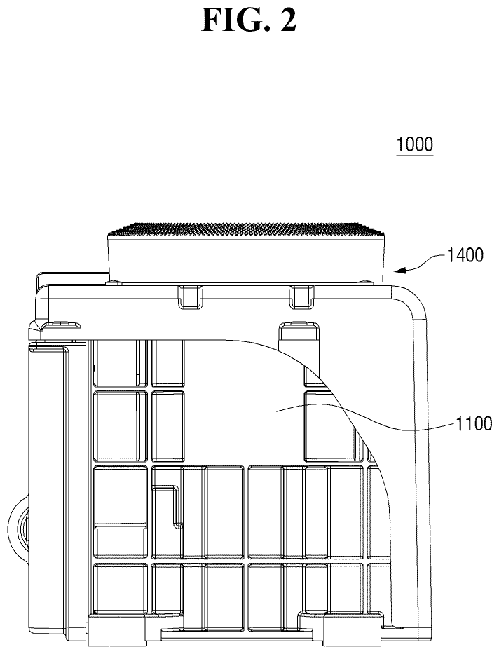

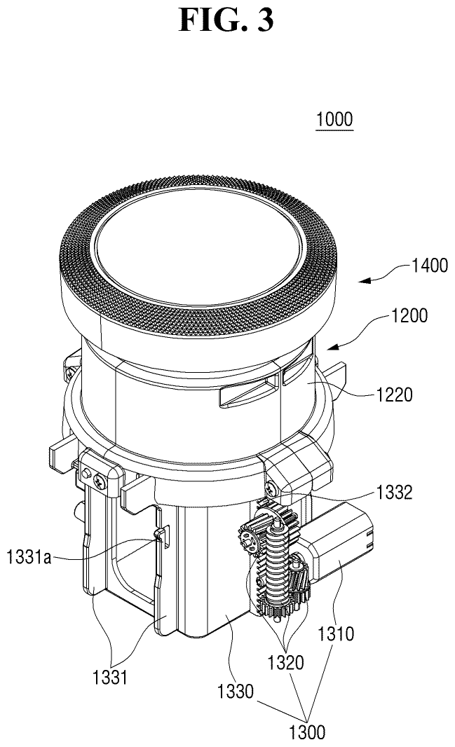

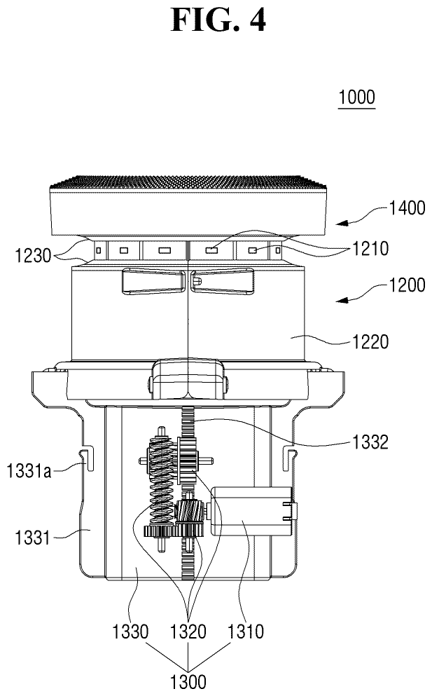

FIG. 1 is a perspective view illustrating the exterior of a sterilization device according to an embodiment of the present disclosure, FIG. 2 is a side view illustrating the exterior of the sterilization device according to an embodiment of the present disclosure, FIG. 3 is a perspective view of the sterilization device according to an embodiment of the present disclosure, and FIG. 4 is a side view of the sterilization device according to an embodiment of the present disclosure. FIGS. 3 and 4 illustrate an exemplary sterilization device with a housing 1100 of FIGS. 1 and 2 omitted for illustration purposes.

Referring to FIGS. 1 through 4, a sterilization device 1000 may include a housing 1100, a sterilization unit 1200, and a driving unit 1300.

The sterilization device 1000 may be used to prevent the spread of various infectious diseases through sterilization of public transportation means, such as buses, taxis, or the like as well as multipurpose facilities, such as buildings, vehicles, elevators, escalators, or the like, allowing for safe and clean utilization. In an embodiment of the present disclosure, for example, the sterilization device 1000 may be installed inside a vehicle, to perform sterilization for the interior space (e.g., cabin space) of the vehicle.

In an embodiment of the present disclosure, for example, the sterilization device 1000 may be installed near the driver's seat, such as in the center console area between the driver's seat and the passenger seat, to enable irradiation of sterilization light in the direction of the dashboard, which can be more heavily contaminated compared to other areas due to frequent contact by the driver. However, the present disclosure is not limited to the dashboard sterilization. The location where the sterilization device 1000 is installed may vary depending on the location of sterilization target areas within the vehicle's interior space.

Furthermore, in an embodiment of the present disclosure, the sterilization device 1000 may be configured as, for example, a popup type that is configured to be moved upward to expose at least a portion of the sterilization unit 1200 outside the housing 1100, when performing sterilization of the vehicle's interior space, and may be configured to be moved downward to dispose at least a portion of the sterilization unit 1200 inside the housing 1100 when it is not in use. However, the present disclosure is not limited to such a configuration. The actual direction in which the sterilization unit 1200 moves for sterilizing the vehicle's interior space may vary depending on the installation location of the sterilization device 1000.

The housing 1100 may include a receiving space therein to accommodate at least a portion of the sterilization unit 1200, and in an embodiment of the present disclosure, the housing 1100 may, for example, accommodate both the sterilization unit 1200 and the driving unit 1300. In an embodiment of the present disclosure, for example, as the sterilization device 1000 may be installed in the center console area, the housing 1100 may be understood as forming a part of the center console area.

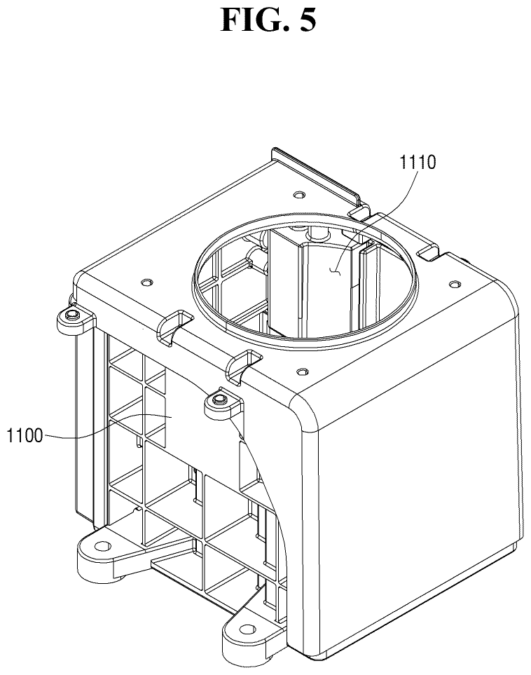

Referring to FIG. 5, the housing 1100 may have an opening 1110 that allows at least a portion of the sterilization unit 1200 to be movable between the interior and exterior of the housing 1100. In an embodiment of the present disclosure, for example, as the sterilization unit 1200 may be moved in a vertical direction, the opening 1110 may be understood as being formed on the top surface of the housing 1100. However, the present disclosure is not limited to such a configuration, and the location where the opening 1110 is formed may vary depending on the direction of movement of the sterilization unit 1200.

Allowing the sterilization unit 1200 to move between the interior and exterior of the housing 1100 through the opening 1110 is to dispose the sterilization unit 1200 outside the housing 1100 when sterilization by the sterilization unit 1200 is needed, and to dispose the sterilization unit 1200 inside the housing 1100 and thereby secure the interior space of the vehicle when sterilization is not required for the vehicle's interior space.

The sterilization unit 1200 may apply sterilization light to at least one sterilization target area for sterilization. As such, the sterilization unit 1200 may include at least one light source 1210 and a mounting part 1220.

The at least one light source 1210 may generate sterilization light with a suitable wavelength and/or intensity for the sterilization function. In an embodiment of the present disclosure, for example, a semiconductor light-emitting element such as a light emitting diode (LED) that emits UV light may be used as the at least one light source 1210, but the present disclosure is not limited thereto. In some such embodiments, the UV light may have a wavelength of about 100 nm to about 400 nm. For example, the UV light may be a short-wave UV (i.e., UV-C), which has a wavelength of about 180 nm to about 280 nm, about 200 nm to about 280 nm, or about 222 nm to about 280 nm. The wavelength or intensity of sterilization light generated from the at least one light source 1210 may vary depending on factors such as sterilization duration or sterilization efficiency.

In an embodiment of the present disclosure, for example, the sterilization unit 1200 may include a plurality of light sources 1210 mounted on the mounting part 1220 along a circumferential direction centered on the direction of movement of the sterilization unit 1200, thereby allowing sterilization of diverse sterilization target areas. However, the present disclosure is not limited thereto. The number of light sources included in the sterilization unit 1200 may vary depending on the number, location, and size of sterilization target areas.

Some of the plurality of light sources 1210 may be arranged at a different spacing from other light sources 1210, and this is because the distance over which sterilization light is irradiated to each sterilization target area from each of the plurality of light sources 1210 or the contamination level of each sterilization target area to be irradiated by the sterilization light from each of the plurality of light sources 1210 may differ. In an embodiment of the present disclosure, some of the light sources 1210 may have a narrower spacing than other light sources 1210, allowing for more uniform sterilization efficiency and sterilization duration even when the light sources 1210 have different sterilization light irradiation distances or there are sterilization target areas with different contamination levels.

For example, some of the plurality of light sources 1210 that have relatively longer irradiation distances to sterilization target areas or are for sterilization target areas with relatively higher contamination levels may be arranged with a closer spacing compared to other light sources 1210, thereby achieving more uniform sterilization efficiency and time required for the sterilization.

Meanwhile, in an embodiment of the present disclosure, for example, sterilization light from each of the plurality of light sources 1210 may be directly applied to each sterilization target area, but the present disclosure is not limited thereto. A reflector 1230, which reflects at least some of the sterilization light generated from each of the plurality of light sources 1210, may be additionally provided to allow appropriate illumination levels of the sterilization light to be applied to appropriate-sized areas of sterilization target areas. Moreover, not only a reflector but also various other types of optical elements such as a mirror, a prism, a lens, or the like, which may affect the amount and path of the sterilization light generated from each of the plurality of light sources 1210, may be used.

When the sterilization unit 1200 includes a reflector, the reflector may have different shapes depending on the location and size of each sterilization target area and the direction and area of light reflected by the reflector. Different shapes of reflectors may be understood as reflective surfaces with different curvatures and/or different inclination angles.

Furthermore, when the sterilization unit 1200 includes a reflector, the reflector may be used individually for each of the plurality of light sources 1210. Alternatively, a single reflector may be used for two or more light sources among the plurality of light sources 1210, or two or more reflectors may be used for one of the plurality of light sources 1210.

The driving unit 1300 may allow the sterilization unit 1200 to move between the interior and exterior of the housing 1100 through the opening 1110 of the housing 1100. In an embodiment of the present disclosure, for example, the sterilization unit 1200 may be moved in the vertical direction by the driving unit 1300. The driving unit 1300 may include an actuator 1310, a gear part 1320, and a support bracket 1330.

The actuator 1310 may generate power for enabling the sterilization unit 1200 to move through the opening 1110 of the housing 1100, and the power of the actuator 1310 may be transmitted to the support bracket 1330 via the gear part 1320, allowing the support bracket 1330 to move in a direction corresponding to the movement direction of the sterilization unit 1200.

In other words, the sterilization unit 1200 may be coupled to the support bracket 1330, and as the support bracket 1330 moves vertically, the sterilization unit 1200 may also move vertically and may thus be able to be disposed inside or outside of the housing 1100.

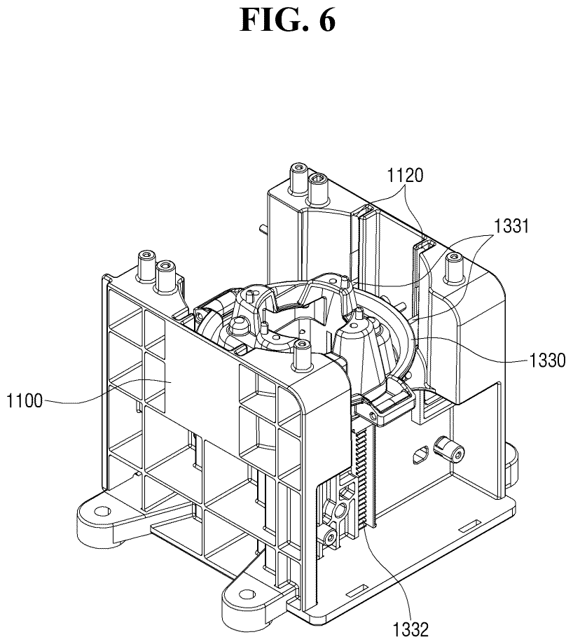

FIG. 6 is a perspective view of a support bracket movably positioned within the housing according to an embodiment of the present disclosure. Specifically, FIG. 6 illustrates a housing 1100 according to an embodiment of the present disclosure, with a portion thereof omitted to show the interior of the housing 1100.

Referring to FIG. 6, the support bracket 1330 may include at least one guide rib 1331 movably inserted into at least one guide rail 1120, which is formed on the inside of the housing 1100 to extend in the movement direction of the sterilization unit 1200. As the at least one guide rib 1331 moves along the at least one guide rail 1120, the movement of the support bracket 1324 may be guided.

If a gap is present in at least one direction between the at least one guide rib 1331 and the at least one guide rail 1120 when the at least one guide rib 1331 moves along the at least one guide rail 1120, unnecessary movement may occur as the sterilization unit 1200 moves. Additionally, the sterilization unit 1200 may descend at a faster rate than the ascending rate, due to the load of the sterilization unit 1200. In this case, even if the same power is transmitted from the driving unit 1300, the movement distance of the sterilization unit 1200 may vary between ascension and descension, making it difficult to accurately control the position of the sterilization unit 1200.

In an embodiment of the present disclosure, for example, a pressurizing member 1130 may be provided to apply pressure to the at least one guide rib 1331 such that the at least one guide rib 1331 and the at least one guide rail 1120 may come into contact. This configuration may prevent unnecessary gap between the at least one guide rib 1331 and the at least one guide rail 1120 in a lateral direction with respect to the movement direction of the sterilization unit 1200 and may ensure that the sterilization unit 1200 has a consistent travel distance during its ascent and descent.

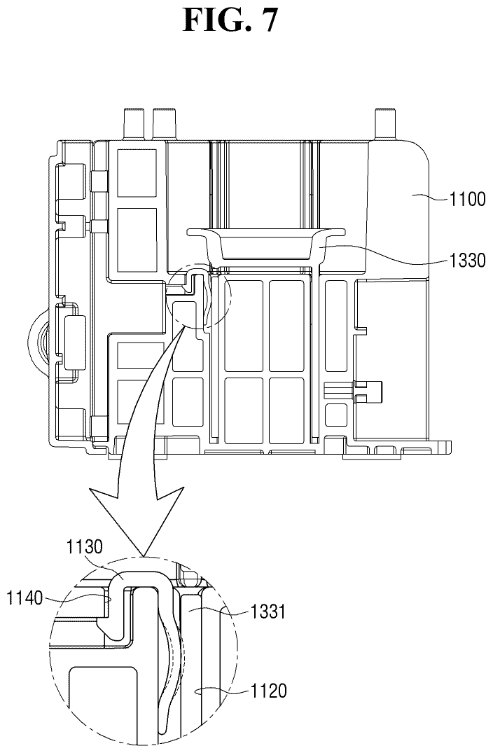

FIG. 7 is a cross-sectional view of a pressurizing member according to an embodiment of the present disclosure. Referring to FIG. 7, a first end portion of the pressurizing member 1130 may be inserted in and coupled to a coupling groove 1140 formed in the housing 1100, and a second end portion of the pressurizing member 1130 may be elastically deformed by the contact with the at least one guide rib 1331. Accordingly, both ends of the at least one guide rib 1331 may be disposed at a predetermined distance in the lateral direction with respect to the movement direction of the support bracket 1330.

In an embodiment of the present disclosure, for example, both end portions of the pressurizing member 1130 may be bent in a substantially “U” shape, but the present disclosure is not limited thereto. The pressurizing member 1130 may have various shapes that can be elastically deformed by the at least one guide rib 1331 and generate a corresponding restorative force.

Thus, when the second end portion of the pressurizing member 1130 is elastically deformed by the at least one guide rib 1331, the restorative force may cause the at least one guide rib 1331 to come into contact with the inner surface of the at least one guide rail 1120. Accordingly, a gap between the at least one guide rib 1331 and the at least one guide rail 1120 may be prevented or minimized.

Additionally or alternatively, an elastic part 1331a, which is disposed to have an end portion thereof in contact with an inner surface of the at least one guide rail 1120, may be formed in the at least one guide rib 1331.

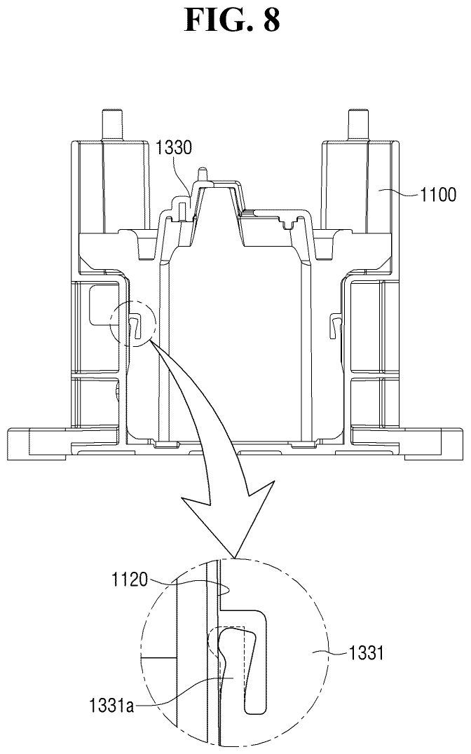

FIG. 8 is a cross-sectional view of the elastic part formed in the support bracket according to an embodiment of the present disclosure. Referring to FIG. 8, the elastic part 1331a may be formed in the guide rib 1331 of the support bracket 1330 to be elastically deformed by the force from the inner surface of the at least one guide rail 1120. When the elastic part 1331a is elastically deformed, unnecessary gap may be prevented between the at least one guide rib 1331 and the at least one guide rail 1120 in the lateral direction with respect to the movement direction of the sterilization unit 1200 due to the restorative force of the elastic part 1331a.

In an embodiment of the present disclosure, for example, the at least one guide rib 1331 and the at least one guide rail 1120 may be formed on opposite sides of the support bracket 1330. In this case, the elastic part 1331a may be formed in each of the guide ribs 1324 on opposite sides of the support bracket 1330.

In the above-described embodiments, the pressurizing member 1130 may be used to prevent a gap in a first direction perpendicular to the movement direction of the support bracket 1330, and the elastic part 1331a may be used to prevent a gap in a second direction perpendicular to the movement direction of the support bracket 1330 and to the first direction. However, the present disclosure is not limited to such a configuration. Either the pressurizing member 1130 or the elastic part 1331a may be used for any of the first and second directions.

For example, in the support bracket 1330, rack gears 1332 may be formed along the movement direction of the sterilization unit 1200. In this example, the gear part 1320 may include one or more pinion gears, allowing the support bracket 1330 to move vertically due to the power of the actuator 1310.

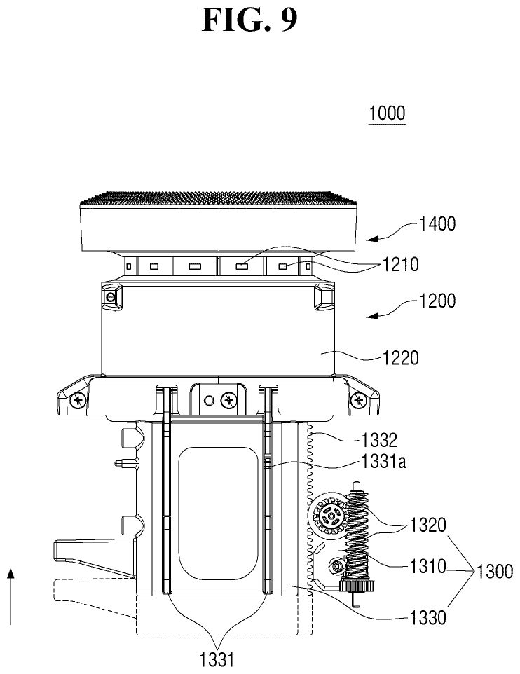

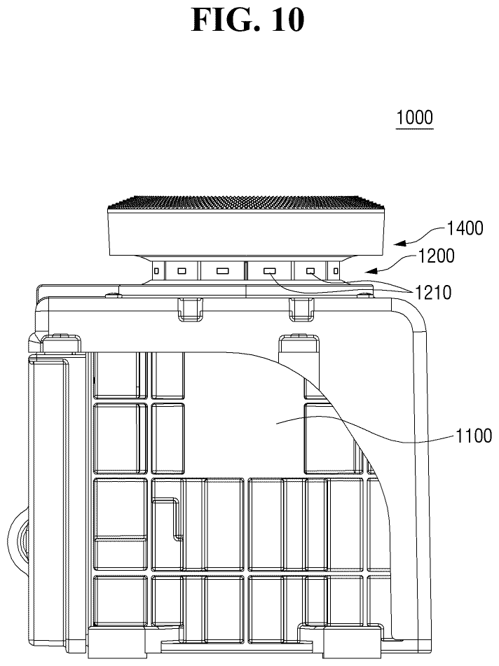

In the sterilization device 1000, when sterilization is needed, power (e.g., a torque) from the actuator 1310 may be transmitted to the support bracket 1330 via the gear part 1320 to move the support bracket 1330 upward, as illustrated in FIG. 9. As a result, as illustrated in FIG. 10, the sterilization unit 1200 may be moved upward to be exposed outside the housing 1100 for irradiating the sterilization light. On the other hand, when sterilization is not needed, the support bracket 1330 may be moved downward so that, as illustrated in FIG. 2, the sterilization unit 1200 is withdrawn within the housing 1100, ensuring that the sterilization unit 1200 does not occupy the interior space of the vehicle when sterilization is not needed.

In an embodiment of the present disclosure, for example, the sterilization unit 1200 may be moved vertically by the driving unit 1300, but the present disclosure is not limited thereto. The sterilization unit 1200 may also rotate about an axis parallel to its movement direction, in which case an additional driving unit for rotating the sterilization unit 1200 may be additionally provided. In this case, the sterilization unit 1200 may be rotated while being exposed outside of the housing 1100, and the additional driving unit for rotating the sterilization unit 1200 may also be supported by the support bracket 1330 to be vertically transportable.

Meanwhile, the sterilization device 1000 may further include an illumination unit 1400, which prevents foreign substances from entering from the outside by covering the opening 1110 of the housing 1100 when the sterilization unit 1200 is disposed within the housing 1100, which may also enhance the exterior appearance by allowing an illumination image having a predetermined shape to be formed therewith.

In an embodiment of the present disclosure, for example, the illumination unit 1400 may be provided on an upper side of the sterilization unit 1200, but the present disclosure is not limited thereto. The illumination unit 1400 may be provided at a location that allows for adequate visibility by the driver.

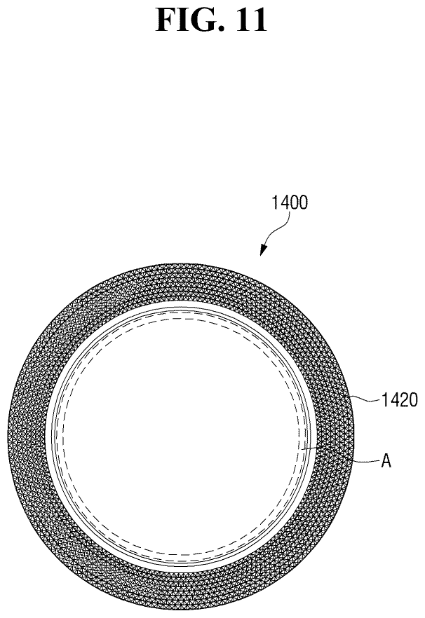

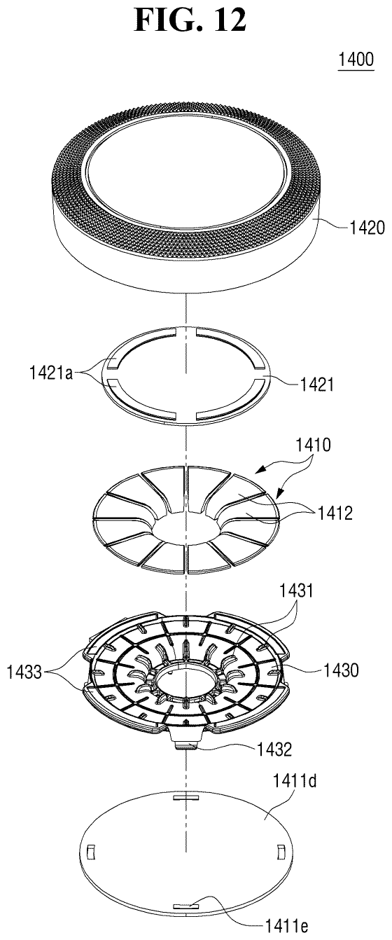

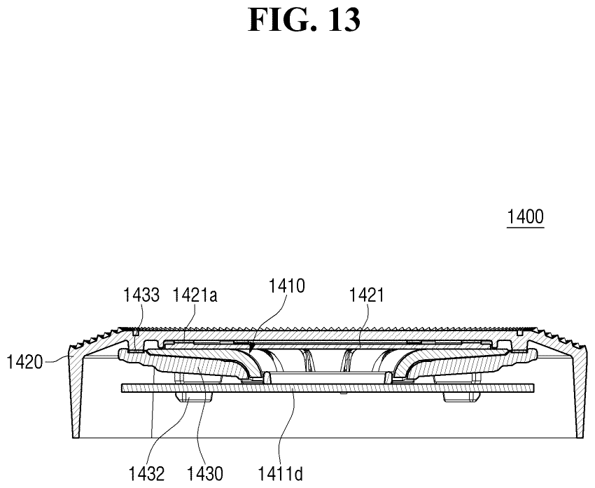

FIG. 11 is a plan view of an illumination unit according to an embodiment of the present disclosure, FIG. 12 is an exploded perspective view of the illumination unit according to an embodiment of the present disclosure, and FIG. 13 is a cross-sectional view of the illumination unit according to an embodiment of the present disclosure.

Referring to FIGS. 11 through 13, the illumination unit 1400 may include at least one light-emitting element 1410 and a light-transmitting cover 1420. The illumination unit 1400 may be configured such that at least some of the light generated from the at least one light-emitting element 1410 passes through the light-transmitting cover 1420 to form an illumination image having a predetermined shape.

In an embodiment of the present disclosure, for example, the illumination unit 1400 may include a plurality of light-emitting elements 1410 arranged in the circumferential direction with respect to the center of the illumination unit 1400 to enable the formation of an approximately circular illumination image, but the present disclosure is not limited thereto. The number and locations of light-emitting elements 1410 may vary depending on the shape of the illumination image to be formed by the illumination unit 1400.

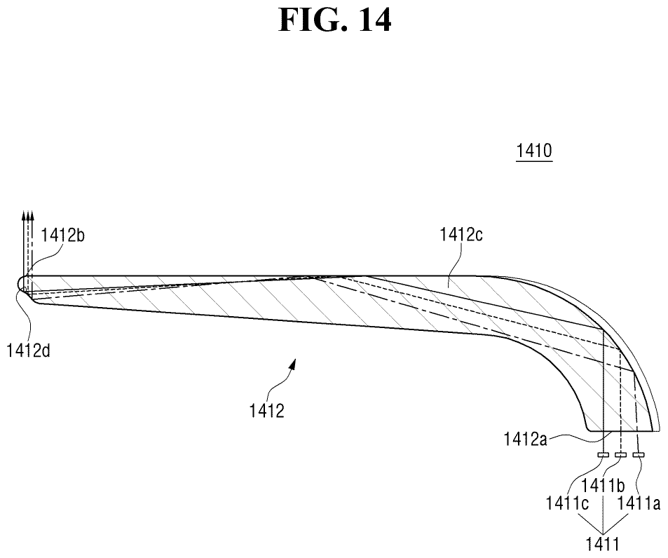

FIG. 14 is a cross-sectional view of a light-emitting element according to an embodiment of the present disclosure. Specifically, FIG. 14 illustrates one of a plurality of light-emitting elements 1410, and the other light-emitting elements 1410 may be configured substantially identically only with some differences in installation positions and/or directions.

Referring to FIG. 14, the illumination unit 1400 may include a light source part 1411 and a light guide part 1412.

The light source part 1411 may include one or more light sources 1411a, 1411b, and 1411c installed on a substrate 1411d. In an embodiment of the present disclosure, for example, the light source part 1411 may include a plurality of light sources 1411a, 1411b, and 1411c that emit light of different colors, such as red light, green light, and blue light, to enable the formation of illumination images in various colors. The light sources 1411a, 1411b, and 1411c will hereinafter be referred to as first, second, and third light sources 1411a, 1411b, and 1411c, respectively, which emit red light, green light, and blue light, respectively.

When the light sources 1411a, 1411b, and 1411c that emit light of different colors are used as the light source part 1411, there is a possibility of color separation occurring due to the differences in the refractive index of each light based on the differences in their wavelengths. The light sources 1411a, 1411b, and 1411c may be disposed differently in consideration of the differences in the refractive index of each light, thereby preventing color separation.

The light guide part 1412 may guide and emit incident light from the light source part 1411. The light guide part 1412 may include an incident portion 1412a for receiving incident light, an emitting portion 1412b for emitting the incident light from the incident portion 1412a, and a transmitting portion 1412c for transmitting the incident light from the incident portion 1412a to the emitting portion 1412b. In an embodiment of the present disclosure, for example, a reflecting portion 1412d, which may reflect the light transmitted by the transmitting portion 1412c toward the emitting portion 1412b, may be formed near the emitting portion 1412b such that the light may be emitted in a direction substantially perpendicular to the direction in which the light is transmitted by the transmitting portion 1412c, through the emitting portion 1412b, which is formed in an upper direction.

The reflecting portion 1412d may be formed with a convex shape toward the direction in which the light is transmitted by the transmitting portion 1412c, and this configure may allow the light emitted through the emitting portion 1412b to be spread relatively wider, ensuring the emission of the light with appropriate brightness. Additionally, surface treatments such as corrosion treatment may be applied to the emitting portion 1412b to allow light with a more uniform brightness to be emitted.

In an embodiment of the present disclosure, for example, the reflecting portion 1412d may be disposed below the emitting portion 1412b to emit the light toward the light-transmitting cover 1420, which is disposed above the light-emitting elements 1410, and to reflect the light transmitted by the transmitting portion 1412c in the upward direction. However, the present disclosure is not limited thereto, and the locations at which the reflecting portion 1412d is formed with respect to the emitting portion 1412b may vary depending on the location of the emitting portion 1412b.

The light guide part 1412 may allow incident light from the incident portion 1412a to be transmitted to the emitting portion 1412b, which is disposed at a greater radial distance from the center of the illumination unit 1400 than the incident portion 1412a. Due to such a configuration, the first light source 1411a may be disposed closest to the center of the illumination unit 1400, and the third light source 1411c may be disposed farthest from the center of the illumination unit 1400. Accordingly, even the light from each of the plurality of light sources 1411a, 1411b, and 1411c has different refractive indices due to differences in wavelength, color separation may be prevented or minimized.

Meanwhile, the plurality of light-emitting elements 1410 may be disposed to be accommodated by a cover bracket 1430, and the cover bracket 1430 may include partition parts 1431, which are for aligning the positions of the light guide parts 1412 of the plurality of light-emitting elements 1410.

Furthermore, coupling protrusions 1432, which are configured to be inserted into and coupled to coupling grooves 1411e formed on the substrate 1411d, may be formed on the cover bracket 1430. Accordingly, the substrate 1411d may be coupled to the cover bracket 1430, and the plurality of light guide parts 1412 may be accommodated by the cover bracket 1430, allowing the positions of the light guide parts 1412 and the cover bracket 1430 to be aligned with each other.

A transparent or translucent region A may be formed in the light-transmitting cover 1420 in an area corresponding to the locations of the emitting portions 1412b, from which light is emitted. The transparent region A may be formed by placing a blocking member 1421, which blocks or obstructs the passage of light, in areas other than the areas where the light is to be emitted from the light-emitting elements 1410.

The blocking member 1421 may be fixed to the inner side of the light-transmitting cover 1420 through fixing members 1421a. In an embodiment of the present disclosure, for example, the fixing members 1421a may be formed as adhesive pads, but the present disclosure is not limited thereto. The blocking members 1421 may be fixed to the light-transmitting cover 1420 not only by adhesive pads, but also by means of hook coupling or screw coupling.

Additionally, coupling members 1433 for coupling with the light-transmitting cover 1420 may be provided on the outer circumference of the cover bracket 1430. In an embodiment of the present disclosure, for example, adhesive pads may also be used as the coupling members 1433, similar to the fixing members 1421a, but the present disclosure is not limited thereto. The cover bracket 1430 and the light-transmitting cover 1420 may be coupled to each other not only by adhesive pads, but also by means of hook coupling or screw coupling.

As mentioned earlier, the transparent region A of the light-transmitting cover 1420 may be formed by the blocking member 1421, but the present disclosure is not limited thereto. Instead of using the blocking member 1421, a colored blocking layer, such as a black blocking layer, may be disposed or coated on the inner surface of the light-transmitting cover 1420 by a process such as deposition or coating. Then, areas of the blocking layer corresponding to the regions where the light is to be emitted from the plurality of light-emitting elements 1410, i.e., areas of the blocking layer corresponding to the emitting portions 1412b of the light guide parts 1412, may be removed through a process such as laser cutting to form the transparent region A. When the transparent region A is formed through laser cutting, may the transparent region A not only pass light therethrough, but also allow an image corresponding to predetermined letters, patterns, or combinations thereof to be formed.

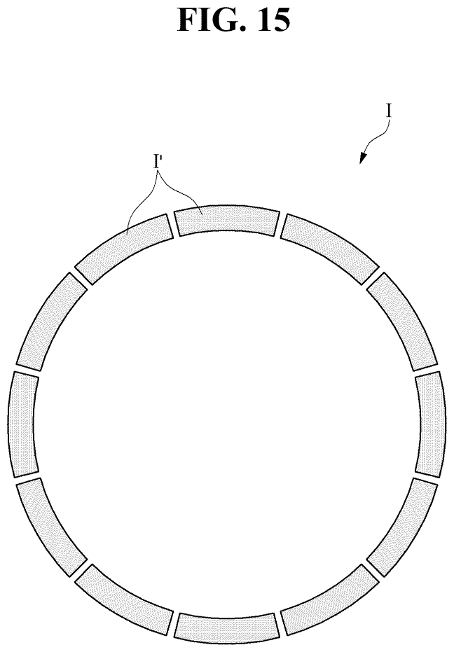

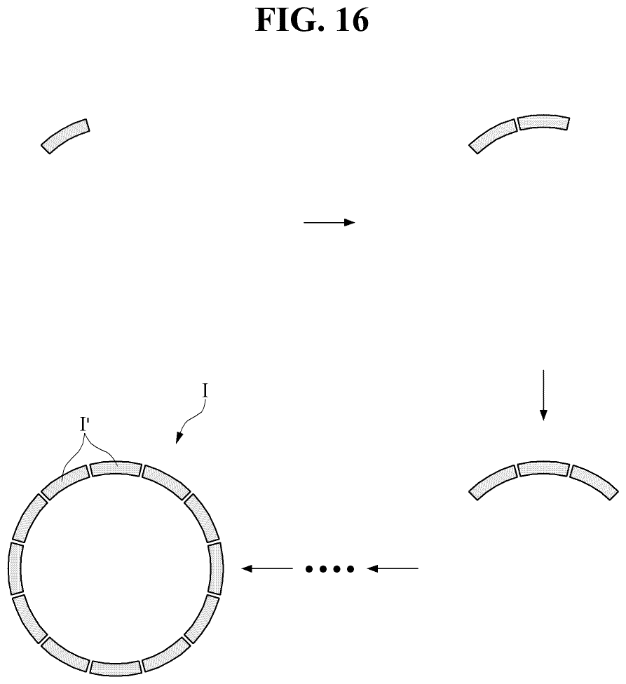

The illumination unit 1400 may enable the implementation of various illumination images depending on the sequence in which the plurality of light-emitting elements 1410 are turned on. For example, referring to FIG. 15, when the plurality of light-emitting elements 1410 are simultaneously turned on, sub-images I′ generated by the light-emitting elements 1410 may be combined together so that an illumination image I having an approximately circular shape may be formed by the illumination unit 1400. Conversely, referring to FIG. 16, when the plurality of light-emitting elements 1410 are sequentially turned on in a particular order, a dynamic illumination image I may be created.

The illumination unit 1400 may be used for a welcome function that provides the impression of the vehicle welcoming the driver when they board, thus enhancing communication between the driver and the vehicle. The illumination unit 1400 may also serve the purpose of indicating at least one of sterilization status or sterilization progress.

For example, when sterilization by the sterilization unit 1200 is complete, all of the plurality of light-emitting elements 1410 may be turned on. Conversely, when sterilization by the sterilization unit 1200 is in progress, some of the plurality of light-emitting elements 1410 may be turned on, while other light-emitting elements 1410 may be turned off, depending on the status of the sterilization progress. In some embodiments, some or all of the plurality of light emitting elements 1410 may be configured to blink.

In a case where the sterilization progress is displayed by sequentially turning on the plurality of light-emitting elements 1410 and the transparent region A of the light-transmitting cover 1420 is formed through laser cutting, a word such as “END” may be displayed in the area corresponding to the light-emitting element 1410 that is last turned on among the plurality of light-emitting elements 1410, within the transparent region A. Such a function may make it easier for the driver to confirm the sterilization status.

As mentioned above, for example, the illumination unit 1400 may include a plurality of light-emitting elements 1410, but the present disclosure is not limited thereto. The cover unit 1400 may also include a single light source, in which case, light generated by the single light source may pass through the transparent region A of the light-transmitting cover 1420 to uniformly form an illumination image.

Furthermore, as mentioned above, for example, the illumination unit 1400 may form an illumination image by turning on the plurality of light-emitting elements 1410, but the present disclosure is not limited thereto. The illumination unit 1400 may also be equipped with a display device such as an organic light emitting diode (OLED) or liquid crystal display (LCD) and may thus be configured to display the aforementioned welcome function, sterilization status, sterilization progress, and other various information that may be provided to the driver using characters, patterns, or combinations thereof.

The sterilization device 1000 may be controlled by a sterilization system that determines at least one sterilization condition and controls a sterilization operation based on the results of the determination.

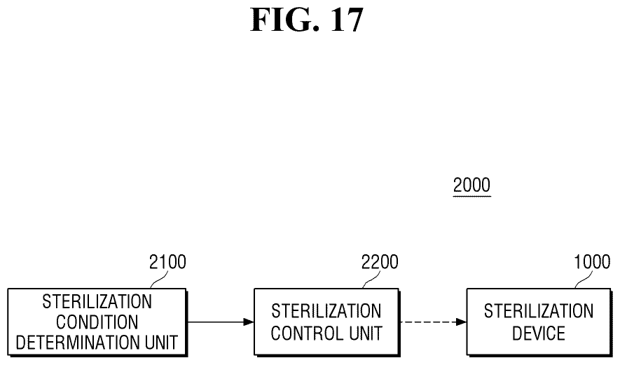

FIG. 17 is a block diagram of a sterilization system according to an embodiment of the present disclosure. Referring to FIG. 17, a sterilization system 2000 may include a sterilization condition determination unit 2100 and a sterilization control unit 2200.

The sterilization condition determination unit 2100 may be configured to determine at least one sterilization condition required for sterilization to be performed.

In an embodiment of the present disclosure, the sterilization condition determination unit 2100 may be configured to determine various sterilization conditions such as ignition status, door open/close status, window open/close status, passenger presence, battery level, temperature, or the like and may be equipped with or operationally connected with various sensors to detect these various sterilization conditions.

The sterilization condition determination unit 2100 may be configured to output an operation signal to the sterilization control unit 2200 when the multiple sterilization conditions are satisfied simultaneously. If sterilization is performed when at least one of the multiple sterilization conditions is not met, it may have adverse effects on humans or increase the risk of accidents during the vehicle operation.

For example, the sterilization condition determination unit 2100 may be configured to output the operation signal only when conditions such as the vehicle's ignition being off, no passenger present, doors closed, battery level above a threshold, and temperature within an acceptable range are met. This is to prevent sterilization from being performed when the vehicle's ignition is on or doors are open, even in the absence of passengers, since there is a likelihood of passengers re-entering the vehicle in such circumstances. Similarly, if the battery level is below the threshold, it may result in issues such as the vehicle being unable to be started, and thus, in such cases, sterilization may be prevented.

Additionally, when the temperature around the space where sterilization is to be performed is outside an acceptable range, issues such as malfunction that may be caused by overheating or failure to satisfy sterilization efficiency or duration requirements may arise. Thus, the sterilization condition determination unit 2100 may ensure that the operation signal is output only when all of the aforementioned sterilization conditions are met concurrently.

The sterilization conditions determined by the sterilization condition determination unit 2100 are not limited to those set forth herein, and may be added, removed, or modified to ensure that no adverse effects are incurred on humans, and at the same time, no problems are caused during the vehicle operation.

When the operation signal is output from the sterilization condition determination unit 2100, the sterilization control unit 2200 may be configured to control the sterilization device 1000 to ensure that sterilization light is irradiated onto at least one sterilization target area.

For example, when the operation signal is output, the sterilization control unit 2200 may be configured to supply power to the driving unit 1300 to move the support bracket 1330 upward and thereby to expose the sterilization unit 1200 to the outside of the housing 1100. Subsequently, electric power may be supplied to the at least one light source 1210 to irradiate the sterilization light to each sterilization target area.

The sterilization control unit 2200 may be configured to perform sterilization according to predefined settings when the operation signal is output from the sterilization condition determination unit 2100.

For example, the predefined settings may include the intensity of sterilization light to be irradiated from the sterilization unit 1200, the irradiation duration of the sterilization light by the sterilization unit 1200, the location of the sterilization unit 1200, or the like. The driver or user may set such settings in advance via input devices such as switches, buttons, touch screens, or the like, installed in the vehicle. The sterilization control unit 2200 ensures that sterilization is performed according to the predefined settings.

In other words, the intensity of sterilizing light may be adjusted differently based on the distance over which the sterilizing light is to be irradiated onto a sterilization target area by the sterilization unit 1200. Additionally, the vertical positioning of the sterilization unit 1200 may be adjusted based on the placement of each sterilization target area. This ensures more uniform sterilization efficiency and sterilization time, even when the irradiation distance of the sterilization light varies or the location of the sterilization target areas shift.

Moreover, the driver may configure the settings in advance for different temperature settings, considering that sterilization efficiency and duration may vary with temperature, and the sterilization control unit 2200 may enable sterilization by adjusting the intensity of the sterilization light, the irradiation duration of the sterilization light, the location of the sterilization unit 1200, or the like, based on the settings for each detected temperature.

In an embodiment of the present disclosure, for example, the sterilization unit 1200 may be movable vertically, and the driver may configure, in advance, settings for the vertical position of the sterilization unit 1200. However, the present disclosure is not limited to this. In a case where the sterilization unit 1200 is configured to rotate, settings for the rotation speed of the sterilization unit 1200 may also be configured in advance.

That is, the sterilization control unit 2200 may be configured to adjust the rotation speed of the sterilization unit 1200 based on temperature and the contamination level of each sterilization target area when the sterilization unit 1200 is rotated. This ensures more uniform sterilization efficiency and sterilization time while preventing malfunction due to overheating.

Meanwhile, the sterilization control unit 2200 may be configured to continuously receive the results of the determination of the sterilization conditions from the sterilization condition determination unit 2100 during the sterilization. If the temperature is outside the acceptable range or the battery level drops below the threshold while sterilization is underway, the sterilization may be stopped or the intensity of sterilization light or the rotation speed of the sterilization unit 1200 may be adjusted to prevent malfunction from overheating and avoid problems such as the vehicle being unable to start.

The results of sterilization performed by the sterilization system 2000 may be displayed through the illumination unit 1400, allowing the driver to more easily check the status of the sterilization and the rate of progress when boarding the vehicle.

In some embodiments, the driver may adjust the predetermined settings and control the sterilization system 2000 via a mobile app executed on a mobile device. In some such embodiments, the mobile device may function as the input device to allow the driver or the user to adjust the settings. In some embodiments, the sterilization condition determination unit 2100 and/or the sterilization control unit 2200 may be provided as software modules of the mobile app, and the mobile device that runs the mobile app may be directly or indirectly connected to the sterilization apparatus 1000 wirelessly or otherwise.

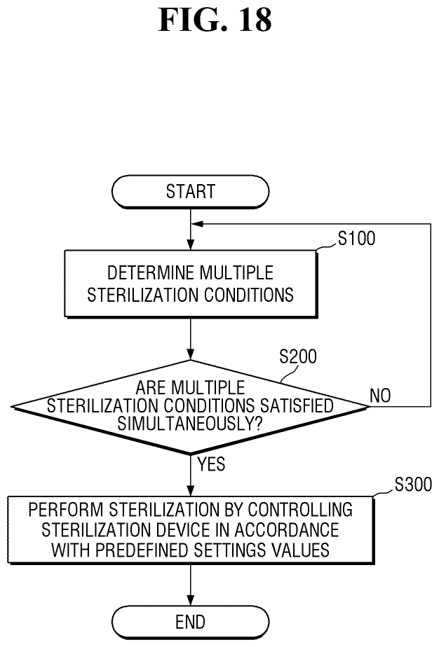

FIG. 18 is a flowchart illustrating an operating method of a sterilization system according to an embodiment of the present disclosure. FIG. 18 depicts an example in which the sterilization condition determination unit 2100 is configured to determine various sterilization conditions for performing sterilization.

Referring to FIG. 18, the operating method involves allowing the sterilization condition determination unit 2100 to determine multiple sterilization conditions (S100) and determine whether the multiple sterilization conditions are satisfied simultaneously (S200).

In step S200, a determination is made as to whether all of the multiple sterilization conditions are satisfied at the same time.

In response to determining that all of the multiple sterilization conditions are satisfied at the same time, the sterilization condition determination unit 2100 may be configured to output an operation signal to the sterilization control unit 2200 for sterilization, and the sterilization control unit 2200 may be configured to control the sterilization device 1000 in accordance with predefined settings to ensure that sterilization is performed on each sterilization target area (S300).

Although exemplary embodiment is described as using a plurality of units such as the sterilization condition determination unit 2100 and the sterilization control unit 2200 to perform the exemplary process, it is understood that the exemplary processes may also be performed by one or plurality of modules. Additionally, it is understood that the terms such as controller, control unit, and determination unit refer to one or more hardware devices that include a memory and a processor. The memory is configured to store the modules and the processor is specifically configured to execute said modules to perform one or more processes which are described herein.

Furthermore, control logic of the present disclosure may be embodied as non-transitory computer readable media on a computer readable medium containing executable program instructions executed by a processor, controller/control unit or the like. Examples of the computer readable mediums include, but are not limited to, ROM, RAM, compact disc (CD)-ROMs, magnetic tapes, floppy disks, flash drives, smart cards and optical data storage devices. The computer readable recording medium can also be distributed in network coupled computer systems so that the computer readable media is stored and executed in a distributed fashion, e.g., by a telematics server or a Controller Area Network (CAN).

It is understood that the term “vehicle” or “vehicular” or other similar term as used herein is inclusive of motor vehicles in general such as passenger automobiles including sports utility vehicles (SUV), buses, trucks, various commercial vehicles, watercraft including a variety of boats and ships, aircraft, and the like, and includes hybrid vehicles, electric vehicles, plug-in hybrid electric vehicles, hydrogen-powered vehicles and other alternative fuel vehicles (e.g. fuels derived from resources other than petroleum). As referred to herein, a hybrid vehicle is a vehicle that has two or more sources of power, for example both gasoline-powered and electric-powered vehicles.

Those skilled in the art to which the present disclosure pertains will understand that the present disclosure can be implemented in various specific forms without changing its technical concept or essential features. Therefore, the embodiments described above are merely exemplary in all aspects and should be understood as non-limiting. The scope of the present disclosure is defined by the claims set forth below, and all modifications or variations derived from the meaning and scope of the claims, as well as the doctrine of equivalents, should be interpreted as being within the scope of the present disclosure.

What is claimed is:

1. A sterilization device comprising:
a housing;
a sterilization unit that irradiates sterilization light for sterilization of at least one sterilization target area; and
a driving unit that moves at least a portion of the sterilization unit between exterior and interior of the housing via an opening provided in the housing.

2. The sterilization device of claim 1, wherein the sterilization unit is moved in a vertical direction through the opening, which is formed on a top surface of the housing.

3. The sterilization device of claim 1, wherein the sterilization unit includes at least one light source to generate the sterilization light and a mounting part in which the at least one light source is mounted.

4. The sterilization device of claim 3, wherein the at least one light source includes a plurality of light sources mounted in the mounting part to be arranged in a circumferential direction centered on an axis that is parallel with a direction in which the sterilization unit is moved.

5. The sterilization device of claim 4, wherein some of the plurality of light sources have a different spacing therebetween from other light sources.

6. The sterilization device of claim 3, wherein the sterilization unit further includes at least one reflector to reflect the sterilization light generated from the at least one light source to the at least one sterilization target area.

7. The sterilization device of claim 6, wherein the at least one reflector has different shapes depending on at least one of an irradiation direction or an irradiation area of the reflected sterilization light.

8. The sterilization device of claim 1, wherein the driving unit includes an actuator, a support bracket coupled to the sterilization unit, and a gear part that transmits power from the actuator to the support bracket, and
wherein, as the support bracket moves, the sterilization unit coupled to the support bracket moves.

9. The sterilization device of claim 8, wherein the support bracket includes at least one guide rib movably inserted in at least one guide rail, which is formed in the housing.

10. The sterilization device of claim 9, further comprising:
a pressurizing member elastically deformed by the at least one guide rib,
wherein a first end portion of the pressurizing member is coupled to the housing, and a second end portion of the pressurizing member exerts pressure to the at least one guide rib to allow the at least one guide rib to contact an inner surface of the at least one guide rail.

11. The sterilization device of claim 9, further comprising:
an elastic part formed to extend from the at least one guide rib,
wherein an end portion of the elastic part is elastically deformed by an inner surface of the at least one guide rail.

12. The sterilization device of claim 1, further comprising:
an illumination unit installed on one side of the sterilization unit to form an illumination image having a predetermined shape.

13. The sterilization device of claim 12, wherein the illumination unit comprises:
at least one light-emitting element that emits light; and
a light-transmitting cover that forms the illumination image by transmitting the light emitted from the at least one light-emitting element therethrough.

14. The sterilization device of claim 13, wherein the at least one light-emitting element comprises:
a light source part; and
a light guide part that guides light incident upon an incident portion thereof from the light source part to be emitted through an emitting portion thereof.

15. The sterilization device of claim 14, wherein the light guide part further includes a transmitting portion for transmitting the incident light from the incident portion to the emitting portion.

16. The sterilization device of claim 15, wherein the light guide part further includes a reflecting portion that reflects the light transmitted by the transmitting portion toward the emitting portion.

17. The sterilization device of claim 13, wherein the at least one light-emitting element includes a plurality of light-emitting elements, and
wherein the plurality of light-emitting elements are turned on simultaneously or sequentially in a predetermined sequence.

18. The sterilization device of claim 1, wherein the sterilization light has a wavelength in a UV-C range.

19. The sterilization device of claim 1, wherein the sterilization light has a wavelength of about 222 nm to about 280 nm.
